(19) United States
(12) Patent Application Publication
(10) Pub. No.: US 2021/0128227 A1
(43) Pub. Date: May 6, 2021

(54) Nocilytic Devices and Methods for Treatment of Dyspareunia

(71) Applicant: Jonathan SACKNER-BERNSTEIN, Pleasantville, NY (US)

(72) Inventor: Jonathan SACKNER-BERNSTEIN, Pleasantville, NY (US)

(21) Appl. No.: 17/095,423

(22) Filed: Nov. 11, 2020

Related U.S. Application Data
(60) Provisional application No. 62/924,361, filed on Oct. 22, 2019.

(51) Int. Cl. A61B 18/14

(52) U.S. Cl. CPC A61B 18/1477; A61B 2018/0022

(57) ABSTRACT

Nocilytic devices and methods for treatment of dyspareunia are provided. In particular, a device is provided, which inserts intravaginally with inflatable balloons to map the location of pain during application of force exerted against/on the luminal side of the vaginal wall and/or vulva of a human patient, or sensors to detect locations for application of nocilytic therapy.

### CROSS-REFERENCE TO RELATED APPLICATIONS

This application claims the benefit of U.S. Provisional Application No. 62/924,361, filed Oct. 22, 2019, the disclosure of which is expressly incorporated by reference herein.

### FIELD OF THE INVENTION

The present invention is directed to treatment of painful vaginal intercourse (dyspareunia), most commonly as manifest in post-menopausal women or those who have received radiation therapy, via devices and methods that block pain signals caused by stretching of the vaginal wall during vaginal intercourse.

### BACKGROUND

Available therapeutic modalities for post-menopausal women suffering from painful intercourse (also known as dyspareunia) often target vaginal dryness, specifically lubricants, moisturizers and hormone replacement therapies. Current treatments of dyspareunia include restoration of hormonal state towards pre-menopausal profile and/or use of lubricants. Lubricants reduce friction without intending for any physiologic effects, such as water, silicone and/or oil-based admixtures, while moisturizers are manufactured with properties expected to affect the vaginal tissues in order to reduce undue friction. Such properties could include pH buffering or trophic cellular effects. Herein, use of the term lubricant encompasses both, as each addresses symptoms related directly to insufficient moistness of the vagina to permit penile-vaginal intercourse without undue friction. Neither sufficiently addresses the symptoms of dyspareunia.

Hormone replacement therapies (locally or systemically administered) theoretically reverse the menopausal changes of the female genitals, (Ballagh 2005; Archer 2010) but serve as an effective treatment of dyspareunia when used intravaginally in only a segment of the affected population and are associated with unacceptable risks when administered systemically.

Although primarily discussed as a treatment for dyspareunia occurring as a result of radiation therapy, mechanical dilation of the vagina is another option. A recent Cochrane Collaboration review failed to show evidence of benefit. (Miles and Johnson 2010). Further, the work of Hsu and colleagues (Hsu et al. 2005) suggests a link between the size of the vagina (increased diameter and cross sectional area) and frequency of pelvic organ prolapse, raising safety concerns about mechanical dilation strategies.

However, these approaches often fail. Imaging of the vagina after menopause in combination with histological studies suggest an alternative mechanism for dyspareunia. With loss of tissue elasticity and narrowing of the vaginal canal in menopause, dyspareunia results from elevated mechanical stress of the vaginal wall stimulating nerve endings of the vagina. In parallel, changes in the vulvar tissue predisposes the external and more superficial genital areas to be susceptible to pain when subjected to the mechanical stress of intercourse. Taught herein are novel methods and tools to block pain from the vagina and/or vulva during intercourse by selective blockade of nociceptive transmission from the vaginal wall and/or vulva.

### SUMMARY OF THE INVENTION

A major cause of pain during vaginal intercourse is due to mechanical forces on the vagina. Taught herein are methods to block the painful signals from the vagina caused by these mechanical forces, along with the necessary tools, referred to herein as neural ablation or neurolysis. These methods and tools block pain from the region of the vagina responsible for the origin of the pain, which does not block pleasurable sensations of intercourse as would be the case if a regional nerve block were employed.

Taught herein are methods for design, manufacture and use of a vaginal/vulvar delivery system for administration of anesthetic and/or neurolytic therapy/therapies to block the signal(s) of pain caused by mechanical forces on the vaginal wall and/or vulva during vaginal intercourse, as well as the specifications for energy, forces and composition of molecules/chemicals delivered by the system disclosed herein.

The example systems described herein utilize a range of tools to selectively ablate the neural receptors in and around the vagina and/or in the vulva, including, as is described herein, pharmacologic neurolytics as well as ultrasound energy and electrical energy, which may be used separately or in combination. Pharmacologic therapies are delivered locally through fenestrations, nozzles or needles, in some embodiments with use of electroporation, iontophoresis and/or mechanical forces to enhance diffusion across the vaginal mucosa and/or vulvar epithelium to reach the target neural receptors.

The embodiments presented include diagnostic functions to enable determination of the site of the pain. Taught herein are methods and tools that are used separately for diagnosis and treatment of dyspareunia, or as a preferred embodiment, used together.

### BRIEF DESCRIPTION OF THE DRAWINGS

FIGS. 1A and 1B are the schematic representations of female genitalia anatomy, as in cross-sectional view in one form (FIG. 1A) and as a line drawing of another form (FIG. 1B).

FIGS. 2A to 2C are examples of intravaginal device as a single segment (FIG. 2A), multi-segment version (FIG. 2B) and device with varying balloon width (FIG. 2C), showing example of device with fenestrations that contain needles in some embodiments.

FIGS. 3A to 3C are examples of intravaginal device with sensors to detect extension and/or flexion. FIG. 3A depicts a bendable intravaginal device without balloons showing channels through which hollow wires are advanced to penetrate vaginal wall. FIG. 3B depicts an intravaginal device with controls and stretch sensors. FIG. 3C depicts an example of the distribution of stretch sensors on the shaft on an intravaginal device.

FIGS. 4A and 4B are examples of the device with retractable needle system, shown with collar covering needles (FIG. 4A) and needles exposed after collar moved (FIG. 4B).

FIG. 5. An example of a needle housing system shown in cross-section.

FIG. 6. An example of a conical screw with guide to deploy and retract needles from housing exemplified in FIG. 5.

FIG. 7. An example of a cross-section of the links between intra-vaginal sub-segments and the connector between the vaginal and remote units.

FIG. 8. An example of articulated link between subsegments of a vaginal insert.

FIG. 9. An example of a device as a single-segment vaginal insert for diagnosis and neuroablation.

FIG. 10. An example of the system with intravaginal and remote connector block, with syringe attached for administration of therapeutic. For clarity, only one channel is shown with connection/syringe.

FIG. 11. An example of the system with intravaginal and remote control, with syringe attached for administration of therapeutic. For clarity, only one channel is shown with connection/syringe.

FIGS. 12A and 12B are examples of the covered intravaginal device with surface stretch sensors. FIG. 12A depicts a preferred embodiment of a multi-segment intravaginal device with geometry to facilitate bending between segments. FIG. 12B depicts an embodiment of the multi-segment device with a covering that includes surface mounted stretch sensors to detect bending of the device.

### DETAILED DESCRIPTION OF THE INVENTION

The Overall Structure of the Device

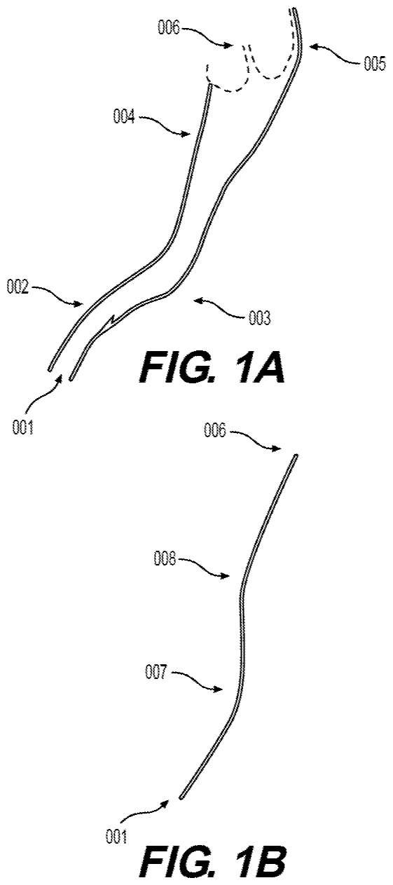

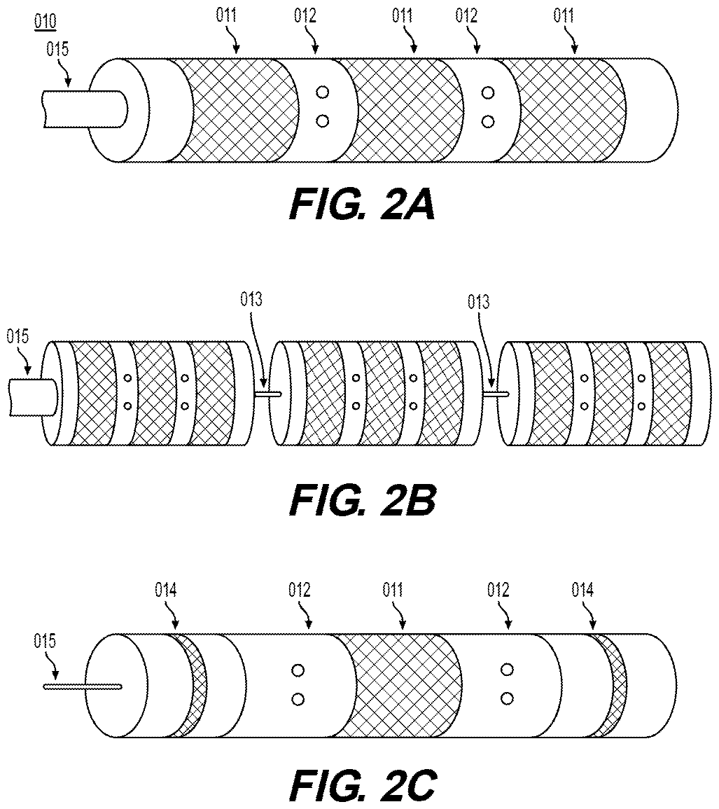

As shown in FIGS. 1A and 2A, the mapping and therapeutic functions are combined into a single device (010), which is inserted through the vaginal introitus (001) and advanced until reaching the posterior vaginal fornix (005). The device is rigid or semi-rigid, with one or more expandable circumferential balloons (011) extending from the proximal to the distal end, each of which is inflated/deflated or filled/emptied independently. These balloons are air filled in preferred embodiments and additional embodiments are liquid filled. In preferred embodiments of the invention, the diameter of the device (010) is between 10 and 150 mm, with preferred embodiments between 18 and 74 mm. (Veale et al. 2015)

The device is a cylinder in one embodiment. In another embodiment of the invention, this inserted device has a shape appropriate for the shape of the human vagina, such as an ellipse or dumbbell cross sectional shape, such as those defined by Pendergrass et al. (Pendergrass et al. 2000) In another embodiment of the invention, the cross sectional shape is a crescent.

A preferred embodiment has a cross-sectional circular geometry, with others ellipsoid or dumbbell shaped. Another embodiment is shaped to approximate the H-like cross sectional shape of the vagina as well as a crescent shaped cross section. (Krantz 2006) Additional embodiments have three dimensional shapes, such as those described by Pendergrass et al (Pendergrass et al. 2000). These shapes can be based on vaginal casting, which may result in shapes such as conical (conical with the posterior fornix being noticeably wider than the anterior canal), parallel sided (long, nearly parallel sides which are only slightly more narrow than the posterior fornix), heart (flattened vagina characterized by a markedly distended posterior fornix constricted at mid-fornix to produce a scallop or heart shape), slug (distended anterior and lateral walls into a distinct bulge or balloon and pumpkin seed (wider from side to side with little depth from anterior to posterior walls).

In a preferred embodiment, the tube is manufactured of medical-grade plastic, and in other embodiments, metal such as titanium. In a preferred embodiment, this inserted device is a single unit (FIGS. 2A and 2C) and in others composed of discrete but connected segments, with the later preferred for women whose vagina has more pronounced bends between the 1^{st}/2^{nd} or 2^{nd}/3^{rd} regions and exemplified by a three-segment device (FIG. 2B). Additional embodiments are up to 10 sub-segments. In each embodiment with multiple parts, these parts are linked by medical grade plastic multi-lumen tubes, with up to 100 lumens for control of balloons, 100 lumens for injection and 100 lumens for control wires, with preferred embodiment containing from 1-50 lumens to control individual balloons, 1-20 lumens for injection into groups of 1-32 needles and 1-10 lumens for control wires, with an exemplar shown for a 44-lumen configuration (FIG. 7). These lumens (021) are used for balloon control, injection of materials, power for visualizing component, images from visualizing component, wires for pressure sensors, wires for electroporation and/or iontophoresis, wires to operate ultrasound, diagnostic and/or therapeutic and/or wires to control needle movement in/out of the device housing and into/from the vagina walls and/or vulva.

Needle deployment and retraction are required in all embodiments of this device/system that use needles. In this embodiment, needle deployment is controlled by a cylindrical screw (301) with a guide (dashed lines 303) on the end of its threads (302).

Imaging studies of the menopausal vagina show that the vaginal canal is not necessarily straight, rather, has bends, (Barnhart et al. 2006; Krantz 2006) represented schematically in cross-sectional view in one form (FIG. 1A) and as a line drawing of another form (FIG. 1B). A bend in the canal may be prominent at the mid-lower vagina (002, 003 and/or 007) and/or at the flexure (004 and/or 008), seen between the vaginal introitus (001) to the cervix (006). These bends are subject to increased physical stress, from translational, radial and circumferential forces, and are amongst the regions that are mapped as primary pain triggers. Taught herein are methods and tools that reduce and/or eliminate neural transmission of the sensation of pain from any parts of the vagina that manifests as dyspareunia.

Segmental neural ablation of the vagina is performed using an intravaginal approach, which enables mapping of the region(s) responsible for the pain followed by the delivery of ablation therapy. In a preferred embodiment, the devices taught herein are used as a system in a two-step process. First, the vagina is mapped to identify the location responsible for the pain of intercourse. Second, the nerve endings in and around the vaginal wall are anesthetized and/or ablated to block and/or blunt the perception of pain during intercourse. The ablation procedure is performed to address the anatomic regions where pain is triggered without ablating sensations experienced during sex. This is accomplished via mapping and corresponding limiting treatment.

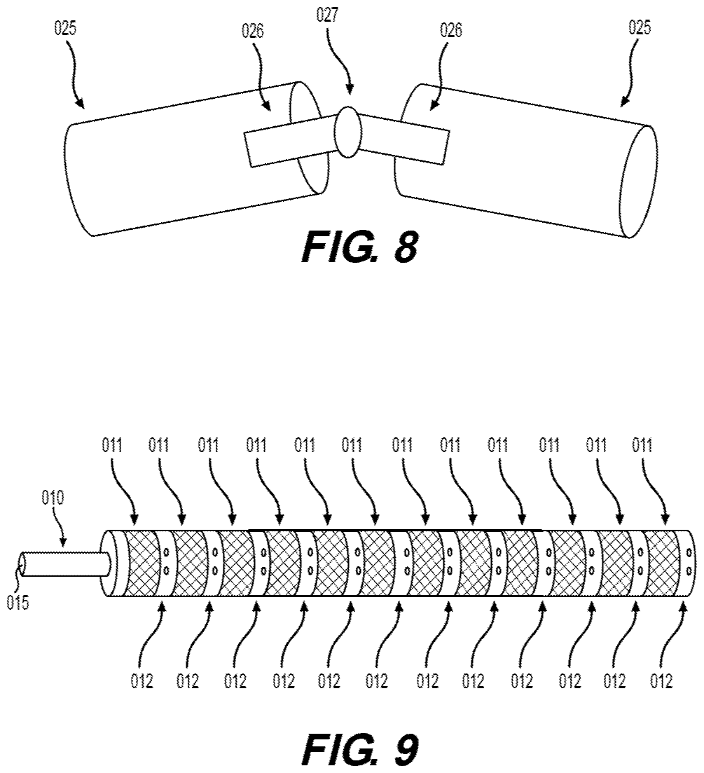

The links between subparts of the insertable device (013) are made of medical grade plastic with capability of bending up to ±20 degrees anteriorly-posteriorly along the longitudinal axis of the vagina. The range of the allowable bend between sub-segments and the diameter of those sub-segments dictate the space required between sub-segments. As shown in FIG. 8, in one embodiment, the multi-lumen subpart connector is constructed with an external hinge (027) connected (026) to each sub-segment (025) to control the direction of flexion/extension as well as serve as a limit of up to ±20 degrees. The links (the assembly of 026 and 027) total 1 cm to 25 cm in length, with preferred embodiments 1 to 5 cm in length.

In a preferred embodiment of the invention, the intravaginal device appears as a single unit externally. Internally the device has from 3 to 15 sub-segments linked as described elsewhere herein. The sub-segments are fabricated with surfaces that are rounded at the edges (016) to allow for close approximation with flexion of up to 20 degrees longitudinally. Otherwise these sub-segments are fabricated as described elsewhere herein. The subsegments are covered by a flexible layer (017) constructed of latex as used in condoms, silicone, fluorosilicones, rubber or any polyurethane, exemplified by Sorbothane™ or other medical grade product, with minimum thickness of 0.1 mm and maximum thickness of 20 mm, though preferred embodiments range from 0.1 to 5 mm, which allows for the needles to penetrate through using the designs taught elsewhere herein before entering into the vaginal wall. The modulus of the materials used for the outer cover in each embodiment are of any magnitude, though each is sufficiently soft to allow for ease of passage of the needle from the inner device or inner sub-segments.

Devices of the present invention can also have one or more features such as balloons, needles, imaging equipment, a cuff, equipment for iontophoresis, electroporation, or other methods of ablation or medicament uptake enhancement.

Balloons

Devices of the present invention have numerous balloons present throughout the device to enable highly accurate mapping of the vagina for pain sensitivity.

FIG. 2 is a preferred embodiment of this device, with low and/or high compliance circumferential balloons (011, 014). In one embodiment, the balloon widths are equal across the device and range from 1 mm to 100 mm. In a preferred embodiment of FIG. 2C, the low compliance balloons are wider than the high compliance balloons, with the former providing high radial and circumferential forces as they inflate during the diagnostic stage of the procedure by inflation via connections remote from the device (030) to pressures ranging from 1 atm to 25 atm. In other embodiments, the high compliance balloons are wider than the low compliance balloons.

The balloon coverage area is as narrow as 1 mm and as wide as 100 mm along the longitudinal axis, and tracks along the entire perimeter in some embodiments and covers a segment of the perimeter in other embodiments, where perimeter refers to the external surface of the device along the radial axis.

The balloon (011, 014) is manufactured from any medical grade material that is stiff, elastic or between those extremes, as is the case for balloons described for use in additional embodiments herein. As examples, the stiff balloons are manufactured of high modulus materials exemplified by non-dispensable poly-paraxylylene and the flexible balloons of low modulus materials exemplified by polydimethylsiloxane (PDMS) or silicone rubber. Use of these materials provides for a range of low to high compliance balloons. In additional embodiments, these materials are combined to provide balloons that expand preferentially in certain directions. In another embodiment, the balloon material is low modulus on the sides and high modulus on top and bottom (viewing the cross section of the intravaginal device), providing a cross-sectional shape that ranges from elliptical to near rectangular when the balloon is inflated. Materials used in additional embodiments for the balloons include polyvinyl chloride, nylons and polyethylenes, with the later including polyethylene terephthalate. The wall thickness of each balloon material is determined by the mechanical and manufacturing issues particular to each, as would be known by one skilled in the art.

Control Unit for the Device

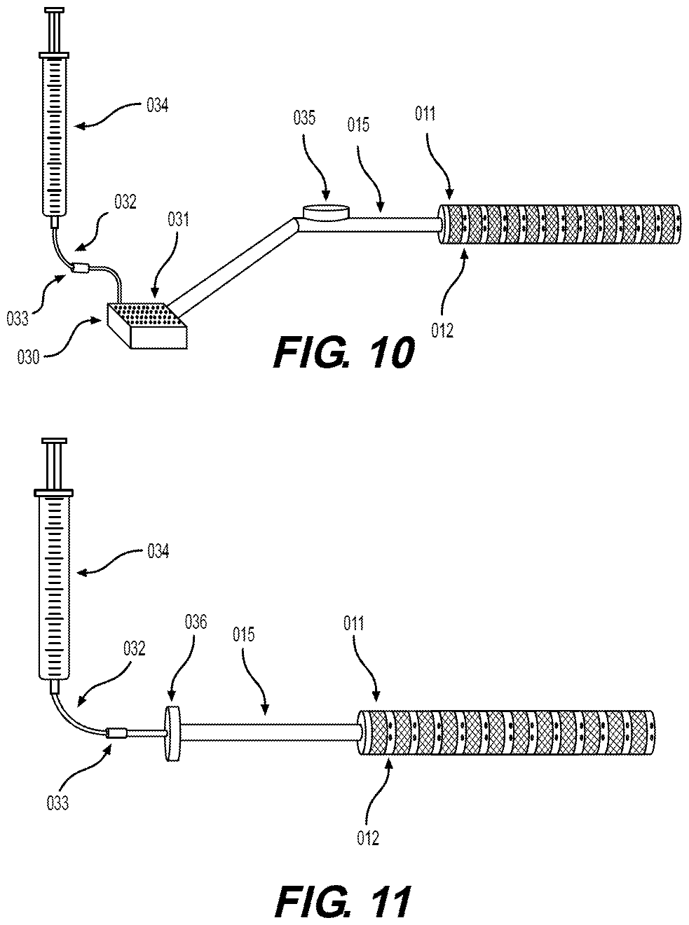

This insertable device is connected (015) to a remote-control device at a distance sufficient for the operator to manage comfortably, which ranges from 0.25 m to 2 m, though additional embodiments are shorter or longer. (FIGS. 10 and 11) This control device includes connectors to permit inflation and deflation of each balloon independently as well as to permit injection into each of the injection sites available on the insertable device that extend from vulva to posterior fornix of the vagina. Luer lock connectors are used, and in a preferred embodiment, needleless connectors are used.

In a preferred embodiment of the invention, the remote-control unit includes one or more of the following components: (a) a calibrated wheel (035) that controls movement of the needles out of the device and into the vaginal wall as well as to retract the needles back into the intravaginal device, (b) a calibrated thumb wheel that controls movement of the needles out of the device and into the vaginal wall as well as to retract the needles back into the intravaginal device, (c) a rotary mechanical selector (036) that aligns any of the needle groups of the intravaginal device with a connector for a syringe, enabling injection into a needle group at each stop in the rotary device, (d) indicator of impedance at the needle tip, (e) wires for connection to imaging system, (f) wires for connection to electrical generator for electroporation and/or iontophoresis and/or (g) hollow wires that end in a needle in the intravaginal device.

In the needleless embodiments, one embodiment of the remote control device is simply a connector block (030) with the channels from the connector (015) spaced on a surface (031) to allow for connection via needleless tubes (032), to which a syringe (034) is connected by commercially available needleless connector (033). Note that in FIG. 10, only one of the channels has a needleless connector shown, in order to provide clearer image. In use, each channel has a needleless connector. This embodiment allows for the syringe (034) to use air or liquid to inflate/deflate a balloon as well as to contain a therapeutic agent exemplified by ethyl alcohol or flushing agent exemplified by saline. The connectors (033) used commercially are known to one skilled in the art, such as those summarized in the literature. (Hadaway 2012)

As in the embodiment of a multi-component insertable device, a preferred embodiment includes a connection between the insertable and control devices made of medical grade semi-rigid plastic, as used in angiographic guiding catheters. This is a multi-lumen connection with up to 100 lumens for control of balloons, 100 lumens for injection and 100 lumens for control wires, with preferred embodiment containing from 1-50 lumens to control individual balloons, 1-20 lumens for injection through needle groups of 1 to 32 needles (or fenestrations) and 1-10 lumens for control wires, with those wires controlling the movement of the microneedles into and for withdrawal from the vaginal walls, the use of imaging component, the use of ultrasound as part of the imaging device and/or for therapeutic use, the use of electrical energy for electroporation and/or iontophoresis. The connection exemplar is shown in FIG. 7 for a 44-channel system. The diameters of the lumens can be equal or vary, with the range from 0.1 mm to 10 mm, allowing air/fluid injections, wire controls, imaging, etc.

In each of these embodiments, the mapping/ablating device inserted into the vagina is connected to a control device, with that connection including channels for balloon control, pharmacologic agent administration, imaging controls, ultrasound controls, needles and/or electrical controls for electroporation/iontophoresis.

These embodiments include control of insertion of the needles into the vaginal wall through controls placed remotely in the operator's hands, through a connection to the intravaginal device. These embodiments include controls to advance the microneedles into the vaginal wall and retract them.

Imaging and Sensors

In additional embodiments of the present invention, the intravaginal device includes device(s) for visual or ultrasonic imaging that are deployed at the distal end in some embodiments and aligned with sites of therapy administration in other embodiments.

In some embodiments are stretch sensors (018) on the surface that detect stress produced by as little as 2 degrees of flexion. These sensors are connected by wires through the lumens described elsewhere herein to the remote-control unit where the operator sees the magnitude and location of stretch on the intravaginal device surface, either visually, graphically, numerically, audibly or through haptic feedback. Area(s) of greatest stretch identify bends in the vagina which are then targeted with neuroablation as taught herein.

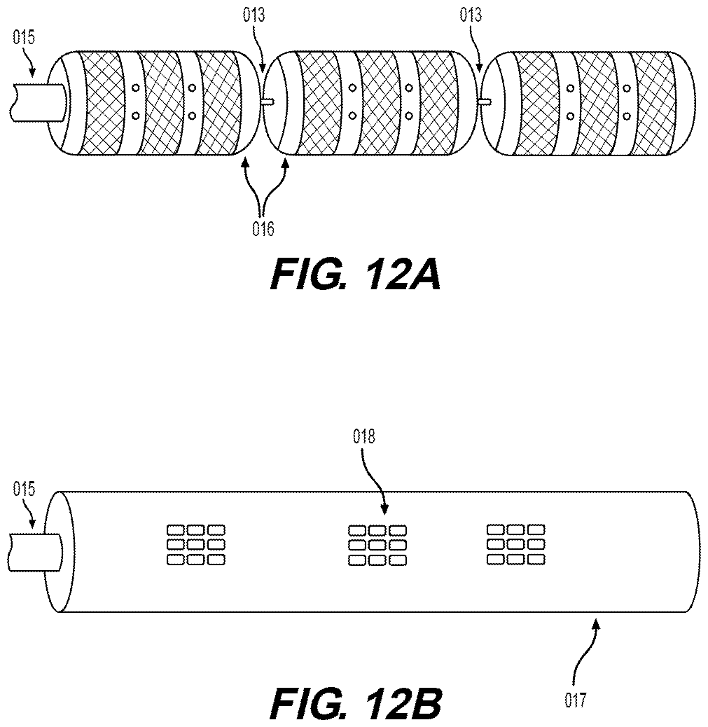

FIG. 12A shows the configuration of a multi-segment intravaginal device, with FIG. 12B showing a preferred embodiment covered and including stretch sensors in a cluster (018) located at the anticipated sites of greatest stretch as predicted by bends in the vaginal canal as shown in FIG. 1. Additional embodiments include distributions across entire surface of the device with sensors arranged in specific or random patterns of varying density.

### Example 1—A Device of the Present Invention without Needles

Administration of medicines using the present invention can also be accomplished without needles.

One embodiment of the present invention is a needleless system. In this embodiment, the fenestrations in the vaginal insert serve as tubes without needles, thus permitting the pharmacologic agent(s) to be injected into the potential space between the device and the vaginal wall. In this embodiment the connection from a syringe connected via Luer lock to a single channel running to the infusion chamber allows for injection from the fenestrations in the surface of the vaginal device (012), as shown in FIG. 2. In this embodiment, balloons are not equal in diameter. A wider balloon (011) from 1 to 100 mm in width is used to test for pain in a particular location. In a region identified as warranting nociceptive fiber ablation, balloon (011) is deflated and balloons (114) surrounding the target region are inflated to segregate the region of interest. In another embodiment, the balloons are equal in width. In another embodiment, the wider balloons are the segregating balloons. The neuroablative pharmacologic therapeutic is then injected into that space via the nozzles (012) and allowed to diffuse through the wall of the vagina. The diffusion can take from 30 seconds to 15 minutes. After that time, an inert fluid such as isotonic or hypotonic saline or similar fluid is injected to dilute the ablative agent. The balloons (014) are deflated as the flushing of the ablative solution continues. In other embodiments, the syringe is connected via a commercially available needleless connector.

An example of this device is shown in FIG. 2C. Using, for example, this type of embodiment of the present invention, the operator inflates balloons most proximal and distal to the balloon that reproduced vaginal pain, creating an isolated segment of the vagina for treatment. A neurolytic agent (alcohol, phenol, carbolic acid, etc.) is introduced into that potential space defined by the surrounding balloons and between the device and vaginal wall, and diffuses through the vaginal mucosa to the adventitial regions where the nerve endings are ablated.

In this embodiment, the operator injects saline, sterile water or other isotonic, relatively neutral pH balanced solution into the space to dilute the neurolytic remaining in the lumen, with this repeated after deflation of the isolating high compliant balloons. The operator has the option to ablate additional regions.

Neural ablation is used across a range of applications, including ablation for focal pain control (Menon and Swanepoel 2010; Zhou, Craig, and Parekh 2015) as well as for nerve endings that serve to drive hypertension. (Gelfand, U.S. Pat. No. 6,978,174) Methods of ablation include application of energy including radio frequency (RF), direct current (DC) and ultrasound (high or low intensity), as well as use of cryoablation tools. Each of these tools produces tissue damage starting at the epithelial/endothelial layer and extends into deeper tissue. In the vagina, that would cause damage to the cell lining that would exacerbate problems caused by loss of lubrication and produce pain during the ablation.

### Example 2—A Device of the Present Invention with Needles

Administration of medicines using the present invention can also be accomplished with needles.

Embodiments of the present invention may include one or more needles for injection.

An alternative is that of Fischell and colleagues (U.S. Pat. No. 9,539,047) who taught use of alcohol injections into a region of nerve endings as a neurolytic strategy to treat hypertension, utilizing intravascular catheters and tools to administer a neurolytic agent into the wall of a blood vessel. In this model, the cells susceptible to the administered pharmacologic ablation agent would be affected at the site of injection and not at the epithelial/endothelial surface.

Shown in this embodiment is a central reservoir (204) within the device which is connected to the remote-control unit via a single channel in the connector (015), and through which the pharmacologic agent is injected. The pharmacologic agent moves into the needle lumens via the hollow base connected centrally (207). Fenestrations (206) in the needles are shown in this embodiment to allow for the pharmacologic agent to be injected through these side holes and the end hole once deployed into the wall of the vagina. Additional embodiments have only an end hole and only side holes.

In the embodiment in FIG. 7, the dashed lines pictured (303) fit into the potential space (207) shown in the needle housing cross-section. These “Ts” (303) fit into grooves at the base of the needles (205) that allow for the needles to be pushed out or pulled in depending on whether the screw (301) is turned clockwise or counterclockwise, respectively. Not shown in this figure is a preferred embodiment with the base of the needle including fenestrations above the T connector to the conical screw, either contiguous or not, to allow the pharmacologic material to enter the needle. The screw is remotely controlled by wire(s) passing through the channels from the remote-control unit as would be familiar to those skilled in the art. This includes a grooved channel (304) to accommodate a wire under sufficient tension to reliably turn the screw in either direction, even against resistance of vaginal wall tissue. In another embodiment, the groove has ridges that match that segment of the wire—providing greater assurance of reliable movement than would be possible if relying on friction alone between two smooth surfaces. Such an assembly includes use of right-angle guides for the wires, and thereby allows wires moving longitudinally to transduce force along the circumferential orientation of the vaginal device.

In another embodiment, needles are fabricated as sharp ends of hollow wires. These wires run from the remote-control device through the connector and into the intravaginal device. In an embodiment, these hollow wires are of one stiffness, with that stiffness sufficient for the needles to penetrate the vaginal wall. In another embodiment, the distal tip is fabricated of stiff metal and the shaft extending to the remote-control unit less stiff material, including metal and plastic in different embodiments. The wires extend into the remote-control unit where the movement of the needles into and out of the vaginal wall is controlled, in some embodiments using a thumb wheel, in others using a thumb slide and in others by direct control of the operator.

The needles of another embodiment advance up to 15 mm in depth in order to infiltrate through to the adventitia surrounding the vagina.

In embodiments with needles entering the vaginal wall at angles other than 90 degrees, the depth of insertion is up to 15 mm, which means that for a needle inserted at 45 degrees, the length of the needle inserted into the vaginal wall is up to 21.2 mm.

In additional embodiments of the invention, needles extend for up to 30 mm at right angles to the device to account for space between the device and the luminal edge of the vaginal wall.

In another embodiment of the invention, needles have electrical conductors at their tips that serve as impedance monitors for detecting contact with the vaginal wall. This impedance sensor is connected via insulated conductor to a wire that travels through a channel or on the surface of the connector that enables measurement by a monitor connected to the remote unit. Embodiments include the impedance sensor composed of copper, platinum, silver, gold, titanium or combinations thereof.

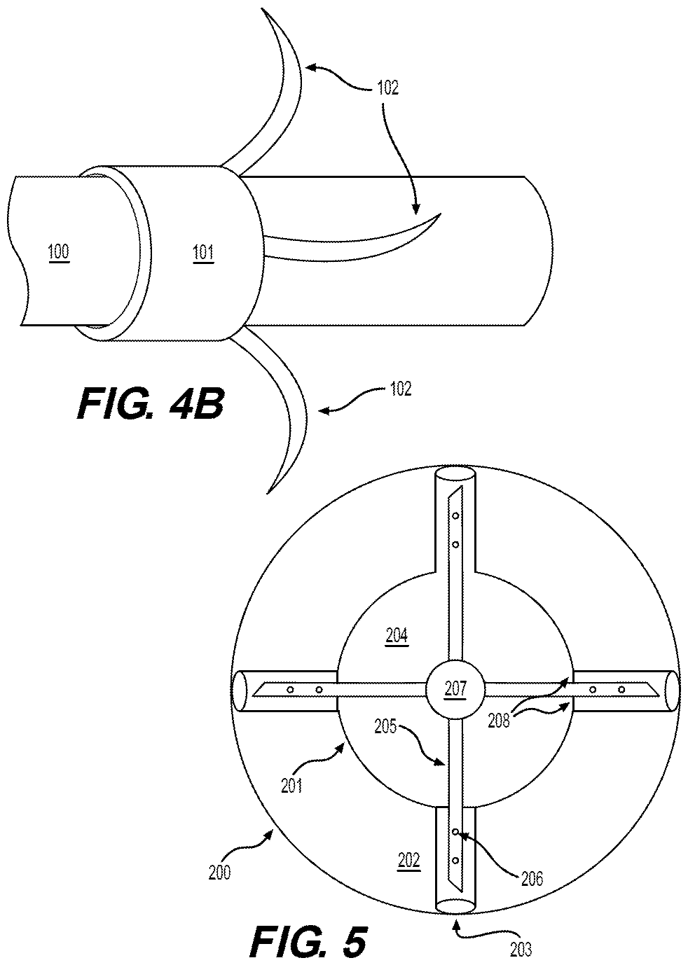

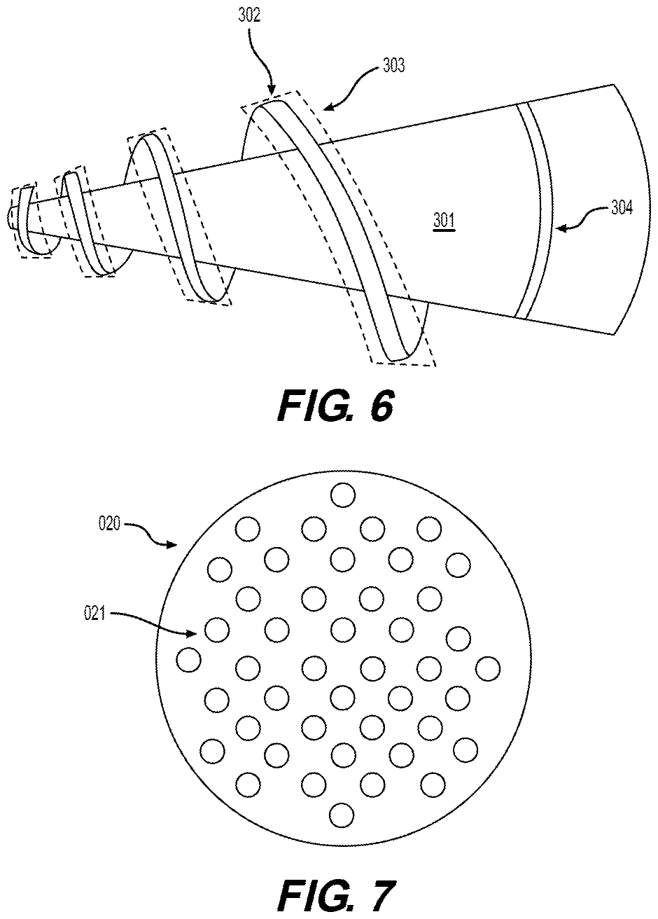

In another embodiment (FIGS. 4A and 4B), the needles (102) are contained under a cuff (101) on the device (100) that slides proximally to uncover them and distally to cover them. As taught by Fischell (U.S. Pat. No. 9,539,047), these needles are made of material that assures memory for them to assume the protruding position (FIG. 4B), as is possible based on use of nitinol metal construction, or in other embodiments a similar springy, memory metal. Upon exposing the needles, the device thumbwheel (035) advances the needle assembly between 0.5 and 10 mm, advancing into the vaginal wall at an angle, providing similar depth as when needles are advanced into the wall at a right angle. The cuff (101) is controlled by pulling a wire to expose the needles and advance the wire to cover the needles. In a preferred embodiment, the thumbwheel is calibrated to allow operator to monitor the length of needle(s) extending from the device. In another embodiment, the needles are advanced up to 35 mm to account for entry into the vaginal wall at an angle other than 90 degrees as well as to account for gap between the device and the vaginal wall.

In another embodiment, a syringe (034) containing a neurolytic agent is connected to administer the therapeutic into the target region that is isolated from the other regions of the vagina by the high compliant balloons forming a cuff. The neurolytic agent is injected by the syringe (034) through the connector (033) into the connector box (031) and then through catheter lumen (021) in the connector (015) into the target vaginal region through needles (205) as shown in the cross-section of the device (200).

The dose of the neurolytic ethanol is from 0.1 to 5 ml from 1 to 100%, with preferred embodiments using solutions of ethanol ranging from 50 to 65%, and for phenol from 1 to 5% solution.

These neurolytic agents and those used as additional embodiments, chosen by one skilled in the art, are administered in their commercially available forms. In additional embodiments, the agents are packaged within liposomes, microsomes and/or nanosomes.

Additional embodiments use anesthetics including lidocaine, bupivicaine, tetracaine, benzocaine and/or related compounds.

Additional embodiments use neurolytics including ethanol, phenol, glycerol, local anesthetics in relatively high concentration, botulinum toxin, guanethidine and/or heated fluids crystalloid solutions.

### Example 3—Treatment using the Present Invention

FIG. 2 shows an embodiment of the intravaginal device (010). In this embodiment, the device has a cylindrical cross section and is surrounded by circumferential balloons. These balloons are inflated and deflated via openings in the surface of the device in a layout subtended by the balloon. In the case of the balloons (011) of FIG. 2 and in other embodiments, these openings are multiple and arranged in lines, grids or other patterns, as well as at random. In various embodiments, the openings have a diameter from 5 microns to 5 mm (these openings are not shown in the figures). These openings are connected a dedicated channel that runs through the connectors (013, 015) to the remote-control unit, where a syringe (034) is connected via needleless connector (033) to that channel (031).

Once the mapping/therapeutic device is inserted into the vagina, the operator connects a syringe to the connector (033) for the target balloon within the control device (030) to inflate that single diagnostic balloon (011, 014) to high pressure in order to reproduce the pain experienced during vaginal intercourse. This is performed sequentially for each of the diagnostic balloons (011) to map where neural ablation is to be performed. The location of balloon inflation that elicits pain is noted for subsequent ablation. Inflation pressures vary as a function of the balloon materials used—and the resulting compliance—with inflation pressures ranging from 1 ATM to 25 ATM in various embodiments and clinical scenarios. In some embodiments pressures are measured within the balloon(s) as well as in the potential space between the balloon and the vaginal wall. Balloons are inflated and then deflated before the next is inflated.

The neuroablation. In a case where a balloon (011) of FIG. 2A elicits pain, ablation of this region of nociceptors in the vaginal wall is accomplished by injecting neurolytic agent into the vaginal wall via the needles concealed in the tubes (012) represented in FIG. 2. The number of needles ranges from 1 to 32 on the surface of the device. A cross-section of an embodiment using a 4-needle configuration is shown in FIG. 5, where the needles (205) and their respective openings (203) are shown equally distributed around the surface perimeter (200). The needles are held within tubes (202) from where they are extended into the vaginal wall and prior to moving/removing the device, retracted into the device. At the base of each tube (202) is a gasket (208) to prevent leakage of the pharmacologic material from the chamber (204). This gasket is sufficiently strong to assure all liquid material travels through the needle (205) and not around the needle.

This second step—the neurolytic ablation—can be accomplished in one or more steps. In a preferred embodiment, needles are inserted into the vaginal wall between 0.5 and 5 mm deep with ethyl alcohol injected as the pharmacologic therapeutic. A total volume of between 0.1 ml and 100 ml is injected, either through a single or multiple needle configuration. A preferred embodiment injects 1-5 ml via each needle. Additional embodiments use phenol at similar doses in place of ethyl alcohol.

An alternative use of the device/system is a multi-step ablation. First, a similar volume of anesthetic, such as lidocaine, xylocaine and other chemical relatives, is injected into the target region, through one or more needles. Needles are retracted and then the balloon that elicited the pain previously is re-inflated. Upon confirmation of reduction in pain, the balloon is deflated, needles redeployed and neuroablative agent administered (ethyl alcohol or phenol). Needles are then retracted, and additional locations treated, as indicated. Upon treatment of any/all regions, needles are retracted, and the device removed from the vagina. This multi-step procedure prevents pain during the procedure that can occur at the time of injection of neuroablative agent.

In the preferred embodiment, the neuroablative agent is injected proximal and distal (012) to the balloon (011) location that identified pain. In FIG. 2A, that would mean that the two sets of needles shown (012) surrounding the middle balloon (011) would be the site of injection. In other embodiments, the vaginal device as more balloons and needle assemblies than shown in FIG. 2A. A preferred embodiment of a single piece has from 3 to 50 balloons, with needle housings (012, 200) between each.

### Example 4—Treatment using a Three-Step Process

Taught herein as a preferred embodiment is a three-step process. First, a device is inserted into the vaginal lumen extending to the posterior fornix, with that device covered by a several circumferential balloons that can be independently inflated/deflated. Second, the operator inflates a balloon one at a time to high enough pressure to generate forces on the vaginal wall (radial and circumferential) until locating the region(s) where the pain of vaginal intercourse is reproduced. Third, a neurolytic is administered into and/or through the vaginal wall by microneedles injecting pharmacologic agent(s) into the vaginal wall, at sites mapped for neuroablation—where balloon inflation produced and/or reproduced pain.

Embodiments of this injection system include one or more needles. A neurolytic agent is injected in that (those) region(s) in a pattern that is circumferential or partially circumferential before the needles are retracted into the device before device removal. These embodiments enable neurolytic agent injection in circumferential or partially circumferential region(s) before the needles are retracted into the device prior to device removal from the vagina.

In additional embodiments, pressure is measured within balloon(s) and/or within the potential space between the intravaginal device and the vaginal wall. In additional embodiments, the neurolytic diffusion across the mucosa is enhanced through electroporation and/or iontophoresis. In additional embodiments, diffusion of the therapeutic agent is enhanced by increasing pressure in the region between the intravaginal device and the vaginal wall by inflating a balloon within that segregated region.

Because the symptoms of dyspareunia may be body position dependent, this embodiment as is case for other embodiments may require use of these tools and methods in particular body positions.

### Example 5—A Device of the Present Invention Employing Iontophoresis & Electroporation

Embodiments include use of electroporation and/or iontophoresis for active diffusion across the vaginal mucosa into and through the vaginal wall, in order to deliver therapeutic agents at higher levels/concentrations more quickly than possible from passive diffusion.

Systems using iontophoresis use pulsed current signals ranging from 0.01 to 10 mA, with voltages from 0.1 to 50 volts, with preferred voltages between 3 and 15 volts. Signal frequency ranges from 10 Hz to 1,000 kHz, with a duty cycle between 1 and 99%.

Embodiments using electroporation use higher voltages, ranging from 10 to 2,000 V/cm, with preferred range between 50 and 500 V/cm. Embodiments are used to match the polar/ionic nature of the therapeutic of interest.

### Example 6—A Device of the Present Invention without Balloons

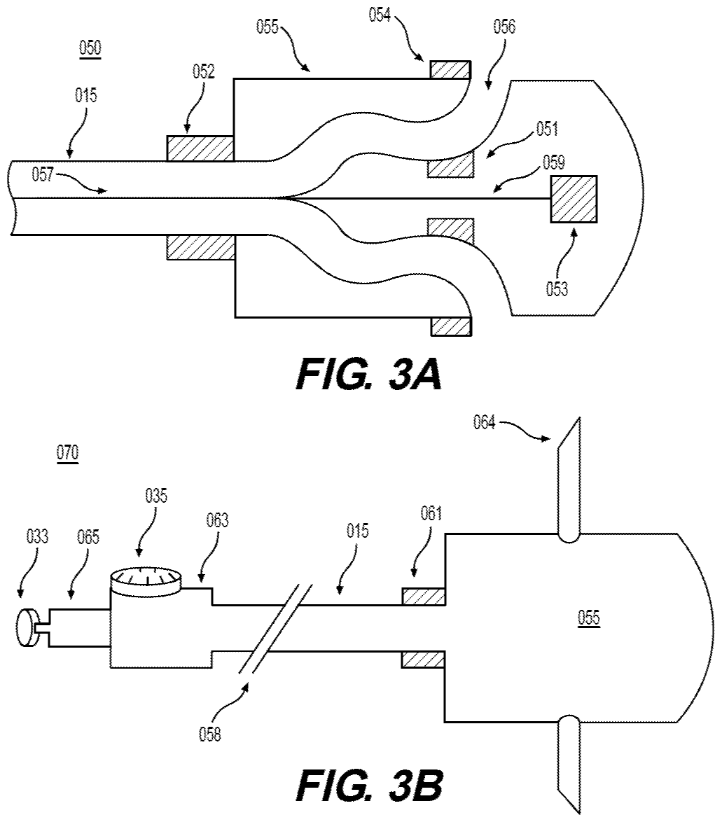

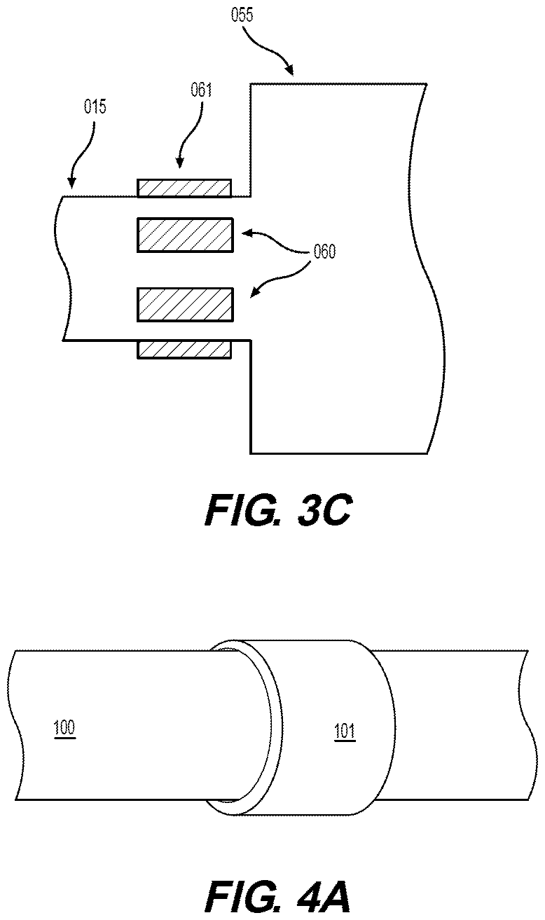

Taught herein as a preferred embodiment is an ablation process without balloons. FIG. 3A shows an embodiment of a device (050). This embodiment detects bends as it is advanced from the vaginal introitus (001) to the posterior fornix (005) along the course of the vaginal canal. This embodiment of the intravaginal device (055) is shown with a cylindrical cross-section, and additional embodiments include cross sectional shapes as described herein, with diameters of preferred embodiments as described herein and length ranging from 10 mm to 150 mm, with preferred embodiments ranging from 25 mm to 100 mm.

On the surface of the device are stretch sensors (054) that detect stress produced by as little as 2 degrees of flexion. These sensors are connected by wires through the lumens described elsewhere herein to the remote-control unit where the operator sees the magnitude and location of stretch on the intravaginal device surface, either visually, graphically, numerically, audibly or through haptic feedback. Area(s) of greatest stretch identify bends in the vagina which are then targeted with neuroablation as taught herein.

FIG. 3 shows a preferred embodiment of the stretch sensors (054) located at the anticipated sites of injection of a therapeutic agent based on the location of the needles within. Additional embodiments include distributions of sensors arranged in specific or random patterns of varying density, though in each case with the stretch sensors aligned with the point along the longitudinal axis and circumferential perimeter where the point of the needle is anticipated to enter the vaginal wall for injection of the therapeutic agent. In some embodiments, the sensors will be offset by a distance which is adjusted for by the bend in the channel (056) through which the hollow wire needle is advanced to assure that the end of the needle is aligned with the sensor.

One embodiment of a stretch sensor is conductive rubber with changes in length proportional to changes in resistance.

In other embodiments of the invention, stretch sensors are replaced with flex sensors, as taught by Langford (U.S. Pat. No. 5,157,372), Beck (U.S. Pat. No. 7,277,004) and others. Such sensors are on the surface in some embodiments (054) and at a flexible segment of the intravaginal device, as shown in FIG. 3 (051) next to the hollow wire needle channel.

In other embodiments, stretch sensors are replaced with accelerometer(s), with preferred a 3-axis accelerometer (053), which detects a change in the path of the intravaginal device as it is inserted and/or withdrawn from the vagina and encounters bends in its course. Shown is an embodiment with an insulated wire conductor (059) leading to the connector (015).

In another embodiment (070) (FIG. 3B) the connector (015) between the control unit (063) and the intravaginal device is not to scale, with indicator of a break (058) in the schematic for a connection that extends beyond lm in a preferred embodiment. The central shaft of the intravaginal device (052) has stretch sensors, with from 1 to 16 sensors aligned along the longitudinal axis capable of detecting 2 degrees of flexion. These sensors are distributed around the central shaft in some embodiments while in others are distributed in any pattern, as shown in FIG. 3C viewed from the top, in order to provide redundant detectors in one or more given direction of flexion. In this preferred embodiment, two sensors are positioned to detect anterior flexion (060) along with two to detect posterior extension (not shown in Figure) with one sensor on each side (061) to detect lateral flexion. In this embodiment as well, the sensors are connected by wires through the lumens described elsewhere herein to the remote-control unit where the operator sees the magnitude and location of stretch on the intravaginal device surface, either visually, graphically, numerically, audibly or through haptic feedback. In this embodiment, the stretch sensors are aligned with the sites where the needles would penetrate the vaginal wall for injection of the neurolytic agent, in terms of the position along the longitudinal axis and circumferential position. The embodiments represented by FIG. 3 show a cross section with only two lumens visible (056). Other lumens allow use of additional hollow wire needles and insulated wires to conduct electricity and/or signals from the intravaginal device to the control unit and/or monitor.

In some embodiments the occurrence of flexion is reflected on the control device (063) when being greater than a minimum amount of flexion (2 degrees or more as a minimum)—as is case for an on/off indicator. In other embodiments the indicator displays the amount of flexion either visually, graphically, numerically, audibly or through haptic feedback.

As shown in FIG. 3, the remote-control device is connected to the intravaginal device as described herein for other embodiments. The handle serving as the remote-control device (063) includes a connector (033) to which a syringe is attached for injecting a therapeutic agent and a thumbwheel (035) with marks to show how far the needle assembly is being advanced into and/or through the vaginal wall.

The embodiment of FIG. 3 shows the housing for the hollow wires (065) to be advanced and retracted using the thumbwheel (035), control unit (063) and the connector (015) to the intravaginal device (055) where the ends of those wires are pointed and sufficiently stiff to serve as needles penetrating into and through the wall of the vagina. These wires are fabricated from a metal such as titanium or stainless steel in preferred embodiments. In other embodiments the length of the wire from the control to the intravaginal device can be a more flexible metal or medical grade plastic, laser welded or glued to the ultimate section that serves as the penetrating needle and its support section with sufficient stiffness to penetrate into and through the vaginal wall.

The hollow wire needles advance as the control wheel (035) turns as part of the housing (065), with those wires moving through channels (057) in the connector (015). These channels (057) curve towards the openings (056) from which the needles (064) extend into the vaginal wall.

The thumbwheel (035) is locked in position that ensures complete retraction of the needles within the intravaginal device. When unlocked, the needle is free to be advanced into and through the vaginal wall by the operator for purpose of injecting the therapeutic agent. The thumbwheel is marked to allow operator to monitor the depth of penetration into and/or through the vaginal wall.

As there are embodiments with specific patterns of distribution of stretch sensors, so too are embodiments described with specific patterns of distribution of needles. In some embodiments, one or more needles are deployable only to the anterior and/or posterior wall(s). In other embodiments needles are deployable only laterally and in others around the entire perimeter.

In this example, the device is advanced from the vaginal introitus towards the posterior fornix. When the device detects a bend, the operator will be notified visually, graphically, numerically, audibly or through haptic feedback of the location of the bend. At that location, the needles are advanced into and/or through the vaginal wall and the therapeutic neuroablative agent is administered as described herein.

Modifications and improvements to the present invention would be known to a person of ordinary skill in the art and are included herein.

1. A device inserted intravaginally with inflatable balloons to map the location of pain during application of force exerted against/on the luminal side of the vaginal wall and/or vulva of a human patient.

2. The device of claim 1, wherein the balloons have high compliance or low compliance.

3. The device of claim 2, wherein the balloons extend around the perimeter of the short axis or one or more balloons cover a segment of at least 10% of the perimeter.

4. The device of claim 3, wherein one or more balloons cover any portion of the perimeter.

5. The device of claim 1, wherein the balloons are constructed of a combination of high and low modulus materials that permit any inflated shape.

6. The device of claim 1, with the balloons connected to a remote-device through a rotary control to provide connection of a single balloon with a syringe.

7. The device of claim 1, wherein the device is any 3-dimensional shape appropriate for insertion into a human vagina, including shapes defined by cross sectional geometry of circle, ellipse, polygon, H-shape, and with three dimensional shapes of rod, cone, parallel sided, heart, slug or pumpkin seed.

8. The device of claim 1, wherein the inflatable balloons extend around the entire perimeter or any portion thereof.

9. The device of claim 2, wherein the balloons are inflatable and deflatable through one or more dedicated channels within the device, to which a syringe/syringes are remotely connected.

10. The device of claim 1 that measures pressure at the potential space between the intravaginal device and vaginal wall.

11. The device of claim 1 that measures pressure within inflated balloon(s).

12. The device of claim 1 that includes imaging component(s).

13. The device of claim 12 with imaging component using ultrasonography.

14. The device of claim 12 with imaging component tuned to the visible spectrum.

15. A device inserted intravaginally with deployable needles to inject a pharmacologic agent into the wall of the vulva and/or vagina.

16. The device of claim 15 with deployable needles connected through dedicated channels within the device, to which a syringe/syringes are remotely connected.

17. The device of claim 15 with the needles connected to a remote-device through a rotary control to provide connection of a needle group with a syringe.

18. A device inserted intravaginally with fenestrations and/or nozzles to infuse a pharmacologic agent to the wall of the vagina for diffusion across the mucosa and into/through the vaginal wall.

19. The device of claim 18 with fenestrations and/or nozzles connected through dedicated channels within the device, to which a syringe/syringes are remotely connected.

20. The device of claim 18 with fenestrations and/or nozzles of any shape including but not limited to circular, ellipsoid, polygonal.
